(19) United States
(12) Patent Application Publication
(10) Pub. No.: US 2009/0191134 A1
(43) Pub. Date: Jul. 30, 2009

(54) Stable aerosol pharmaceutical formulations

(75) Inventors: Rajesh Sawant, Mumbai (IN); Sunil Parab, Goa (IN)

Correspondence Address: CONLEY ROSE, P.C., 5601 GRANITE PARKWAY, SUITE 750, PLANO, TX, 75024, US

(73) Assignee: Medispray LaboratoriesPvt. Ltd., Ponda, IN (US)

(21) Appl. No.: 12/304,363

(22) PCT Filed: Jun. 12, 2007
(86) PCT No.: PCT/GB07/02183

§ 371 Date: Mar. 25, 2009

(30) Foreign Application Priority Data
Jun. 12, 2006 (IN) 918/MUM/2006

(51) Int. Cl. A61K 9/12; A61P 11/06

(52) U.S. Cl. 424/45

(57) ABSTRACT

The present invention provides a stable aerosol pharmaceutical formulation of a beta-agonist, an anticholinergic, or a combination thereof in combination with a cosolvent and optionally a surfactant. The invention also provides a method of making the stable aerosol pharmaceutical formulation and methods of treating bronchoconstriction, asthma and related conditions with the stable aerosol pharmaceutical formulation of the present invention.

### CROSS-REFERENCE TO RELATED APPLICATIONS

This application is a filing under 35 U.S.C. 371 of International Application No. PCT/GB2007/002183 filed Jun. 12, 2007, entitled “Stable Aerosol Pharmaceutical Formulations,” claiming priority of Indian Patent Application No. 918/MUM/2006 filed Jun. 12, 2006, which applications are incorporated by reference herein in their entirety.

### BACKGROUND OF THE INVENTION

Asthma is chronic inflammatory disease affecting about 20 million to 35 million persons worldwide, in which the patient suffers episodes of reversible airway obstruction. Asthma is generally treated by administration of anti-inflammatory drugs or bronchodilators. Anti-inflammatory drugs useful for the treatment of asthma include corticosteroids, mast cell stabilizers, and leukotriene inhibitors. Bronchodilators include beta-agonists, anticholinergics, and methylxanthines. Beta-agonists can be used to treat exercise-induced asthma. Beta-agonists can be combined with other classes of drugs like corticosteroids, anti-cholinergics and leukotriene inhibitors. The combination of a beta-agonist, such as albuterol, and an anticholinergic, such as ipratropium, has proven to be highly effective because the drugs provide bronchodilation by different mechanism of action.

Many asthma drugs are administered through inhalation. Suitable inhalation devices include metered dose inhalers (“MDIs”), dry powder inhalers and nebulizers. Metered dose inhalers conventionally contain one or more liquefied chlorofluorocarbons (“CFCs”) as propellant. Such materials are suitable for use in such applications since they have the right vapor pressures (or can be mixed in the right proportions to achieve a vapor pressure in the right range) and are essentially taste and odor-free.

One such CFC-containing metered dose inhaler is Combivent® Inhalation Aerosol which contains a microcrystalline suspension of ipratropium bromide and albuterol sulfate in a pressurized metered-dose aerosol unit for oral inhalation administration.

Due to environmental concerns, it is now desirable to use hydrofluoroalkane (“HFA”) propellants instead of CFCs in metered dose inhalers containing asthma drugs. For example, US20040184994 relates to a formulation comprising water in an amount of about 0.13 to about 0.18 percent (w/w) of the product formulation, at least one HFA as a propellant, one or more active ingredients and one or more excipients, wherein the preferred active ingredients are ipratropium and albuterol and the excipients are citric acid, ethanol and polyvinylpyrrolidone (“PVP”).

US20050085445 relates to a metered-dose aerosol inhaler composition, which contains a) at least one pharmaceutical active ingredient, b) at least one propellant (preferred propellants being HFA 227 and HFA 134a), c) at least one native or modified cyclodextrin, d) at least one hydrophilic additive, and e) optionally ethanol.

US20050089478 relates to a formulation comprising formoterol and budesonide for use in the treatment of respiratory diseases. The composition further contains HFA-227 propellant, PVP and polyethylene glycol (“PEG”), preferably PVP K25 and PEG 1000.

However, MDI formulations containing HFA propellants do not have suspension characteristics as good as those formulations containing CFC Propellants. For example, an MDI formulation containing the beta-agonist albuterol sulfate and an anticholinergic agent, such as ipratropium bromide or tiotropium, with an HFA propellant is not a stable suspension and either quickly sediments or forms an emulsion.

For this reason, it is difficult to obtain a stable suspension using an HFA propellant. As a result, cosolvents such as water and alcohols (ethanol) have been used in combination with beta-agonist agents and anticholinergic agents in formulations containing HFA propellants. Unfortunately, the use of these cosolvents also leads to stability problems and other issues, such as agglomeration/increased particle size of the active agents, which are also undesirable.

### SUMMARY OF THE INVENTION

According to a first aspect of the present invention, there is provided a pharmaceutical formulation comprising: an anticholinergic agent or a pharmaceutically acceptable salt, pharmaceutically acceptable solvate or pharmaceutically acceptable hydrate thereof; a beta-agonist agent or a pharmaceutically acceptable salt, pharmaceutically acceptable solvate or pharmaceutically acceptable hydrate thereof; a cosolvent; and a hydrofluoroalkane propellant. The anticholinergic agent may also be a pharmaceutically acceptable enantiomer, pharmaceutically acceptable derivative, pharmaceutically acceptable polymorph or pharmaceutically acceptable prodrug thereof. The beta-agonist agent may also be a pharmaceutically acceptable enantiomer, pharmaceutically acceptable derivative, pharmaceutically acceptable polymorph or pharmaceutically acceptable prodrug thereof.

In an embodiment, the cosolvent is selected from the group consisting of polyethylene glycol, propylene glycol, glycerol, and isopropyl myristrate.

The formulation may further comprise a surfactant. The surfactant may be selected from the group consisting of polyvinylpyrrolidone, sorbitan trioleate, oleic acid, citric acid, and polyoxyethylene(4)lauryl ether.

In an embodiment, the anticholinergic agent is selected from ipratropium or tiotropium or pharmaceutically acceptable salts, pharmaceutically acceptable solvates or pharmaceutically acceptable hydrates thereof. Alternatively, the anticholinergic agent is selected from ipratropium or tiotropium or pharmaceutically acceptable enantiomers, pharmaceutically acceptable derivatives, pharmaceutically acceptable polymorphs or pharmaceutically acceptable prodrugs thereof. Suitably, the anticholinergic agent is ipratropium.

In an embodiment, the beta-agonist is selected from the group consisting of albuterol, formoterol, levalbuterol, pirbuterol and salmeterol, or pharmaceutically acceptable salts, pharmaceutically acceptable solvates or pharmaceutically acceptable hydrates thereof. Alternatively, the beta-agonist is selected from the group consisting of albuterol, formoterol, levalbuterol, pirbuterol and salmeterol, or pharmaceutically acceptable enantiomers, pharmaceutically acceptable derivatives, pharmaceutically acceptable polymorphs or pharmaceutically acceptable prodrugs thereof. Suitably, the beta-agonist is albuterol.

In another embodiment, the anticholinergic agent is ipratropium or a pharmaceutically acceptable salt, pharmaceutically acceptable solvate or pharmaceutically acceptable hydrate thereof and the beta-agonist is albuterol or a pharmaceutically acceptable salt, pharmaceutically acceptable solvate or pharmaceutically acceptable hydrate thereof. Alternatively, the anticholinergic agent is ipratropium or a pharmaceutically acceptable enantiomer, pharmaceutically acceptable derivative, pharmaceutically acceptable polymorph or pharmaceutically acceptable prodrug thereof. Alternatively, the beta-agonist is albuterol or a pharmaceutically acceptable enantiomer, pharmaceutically acceptable derivative, pharmaceutically acceptable polymorph or pharmaceutically acceptable prodrug thereof.

In an embodiment, the cosolvent is present in an amount of about 0.05% to about 15% of the total weight of the formulation.

In another embodiment, the surfactant is present in an amount of about 0.00001% to about 10% of the total weight of the formulation.

Preferably, the formulation is substantially free of alcohol.

Preferably, the formulation is substantially free of water.

In an embodiment, the formulation contains less than about 0.1% water by weight of the formulation.

The formulation may comprise ipratropium bromide or its monohydrate, albuterol sulfate, about 0.05% to about 1% polyethylene glycol, about 0.00001% to about 0.1% polyvinylpyrrolidone, and a hydrofluoroalkane propellant.

According to a second aspect of the present invention, there is provided a pharmaceutical formulation comprising: an anticholinergic agent or a pharmaceutically acceptable salt, pharmaceutically acceptable solvate or pharmaceutically acceptable hydrate thereof; a beta-agonist agent or a pharmaceutically acceptable salt, pharmaceutically acceptable solvate or pharmaceutically acceptable hydrate thereof; a polysorbate; and a hydrofluoroalkane propellant. The anticholinergic agent may also be a pharmaceutically acceptable enantiomer, pharmaceutically acceptable derivative, pharmaceutically acceptable polymorph or pharmaceutically acceptable prodrug thereof. The beta-agonist agent may also be a pharmaceutically acceptable enantiomer, pharmaceutically acceptable derivative, pharmaceutically acceptable polymorph or pharmaceutically acceptable prodrug thereof.

The polysorbate may be selected from polyoxyethylene sorbitan monolaurate, polyoxyethylene sorbitan monopalmitate, polyoxyethylene sorbitan monostearate, polyoxyethylene sorbitan monooleate and polyoxyethylene sorbitan monoisostearate. Suitably, the polysorbate may be selected from polyoxyethylene (20) sorbitan monolaurate (Tween 20), polyoxyethylene (20) sorbitan monopalmitate (Tween 40), polyoxyethylene (40) sorbitan monostearate (Tween 60), polyoxyethylene (20) sorbitan monooleate (Tween 80) and polyoxyethylene (20) sorbitan monoisostearate (Tween 120). Suitably, the polysorbate is polyoxyethylene sorbitan monolaurate, for example polyoxyethylene (20) sorbitan monolaurate.

In an embodiment, the polysorbate, for example polyoxyethylene sorbitan monolaurate, is present in an amount of about 0.05% by weight of the formulation.

In another embodiment, there is no cosolvent present in the formulation.

In an embodiment, the anticholinergic agent is selected from ipratropium or tiotropium or pharmaceutically acceptable salts, pharmaceutically acceptable solvates or pharmaceutically acceptable hydrates thereof. Alternatively, the anticholinergic agent is selected from ipratropium or tiotropium or pharmaceutically acceptable enantiomers, pharmaceutically acceptable derivatives, pharmaceutically acceptable polymorphs or pharmaceutically acceptable prodrugs thereof. Suitably, the anticholinergic agent is ipratropium.

In an embodiment, the beta-agonist is selected from the group consisting of albuterol, formoterol, levalbuterol, pirbuterol and salmeterol, or pharmaceutically acceptable salts, pharmaceutically acceptable solvates or pharmaceutically acceptable hydrates thereof. Alternatively, the beta-agonist is selected from the group consisting of albuterol, formoterol, levalbuterol, pirbuterol and salmeterol, or pharmaceutically acceptable enantiomers, pharmaceutically acceptable derivatives, pharmaceutically acceptable polymorphs or pharmaceutically acceptable prodrugs thereof. Suitably, the beta-agonist is albuterol.

In another embodiment, the anticholinergic agent is ipratropium or a pharmaceutically acceptable salt, pharmaceutically acceptable solvate or pharmaceutically acceptable hydrate thereof and the beta-agonist is albuterol or a pharmaceutically acceptable salt, pharmaceutically acceptable solvate or pharmaceutically acceptable hydrate thereof. Alternatively, the anticholinergic agent is ipratropium or a pharmaceutically acceptable enantiomer, pharmaceutically acceptable derivative, pharmaceutically acceptable polymorph or pharmaceutically acceptable prodrug thereof. Alternatively, the beta-agonist is albuterol or a pharmaceutically acceptable enantiomer, pharmaceutically acceptable derivative, pharmaceutically acceptable polymorph or pharmaceutically acceptable prodrug thereof.

Preferably, the formulation is substantially free of alcohol.

Preferably, the formulation is substantially free of water.

In an embodiment, the formulation contains less than about 0.1% water by weight of the formulation.

According to a third aspect of the present invention, there is provided a method of making a pharmaceutical formulation comprising: (a) mixing a hydrofluoroalkane propellant with a cosolvent to form a solution; (b) forming a first homogenized suspension of an anticholinergic agent or a pharmaceutically acceptable salt, pharmaceutically acceptable solvate or pharmaceutically acceptable hydrate thereof; a beta-agonist agent or a pharmaceutically acceptable salt, pharmaceutically acceptable solvate or pharmaceutically acceptable hydrate thereof; and a hydrofluoroalkane propellant; and (c) adding the first homogenized suspension to the solution to form a second homogeneous suspension. The anticholinergic agent may also be a pharmaceutically acceptable enantiomer, pharmaceutically acceptable derivative, pharmaceutically acceptable polymorph or pharmaceutically acceptable prodrug thereof. The beta-agonist agent may also be a pharmaceutically acceptable enantiomer, pharmaceutically acceptable derivative, pharmaceutically acceptable polymorph or pharmaceutically acceptable prodrug thereof.

In an embodiment, the cosolvent is selected from the group consisting of polyethylene glycol, propylene glycol, glycerol, and isopropyl myristrate.

The method may further comprise dissolving a surfactant in the cosolvent. The surfactant may be selected from the group consisting of polyvinylpyrrolidone, sorbitan trioleate, oleic acid, citric acid, and polyoxyethylene(4)lauryl ether.

The method may be for making the formulation described in the first two aspects of the invention.

According to a fourth aspect of the present invention, there is provided a method of making a pharmaceutical formulation comprising: (a) dissolving polyoxyethylene sorbitan monolaurate in a hydrofluoroalkane propellant to form a solution; (b) forming a first homogenized suspension of an anticholinergic agent or a pharmaceutically acceptable salt, pharmaceutically acceptable solvate or pharmaceutically acceptable hydrate thereof; a beta-agonist agent or a pharmaceutically acceptable salt, pharmaceutically acceptable solvate or pharmaceutically acceptable hydrate thereof; and a hydrofluoroalkane propellant; and (c) adding the first homogenized suspension to the solution to form a second suspension. The anticholinergic agent may also be a pharmaceutically acceptable enantiomer, pharmaceutically acceptable derivative, pharmaceutically acceptable polymorph or pharmaceutically acceptable prodrug thereof. The beta-agonist agent may also be a pharmaceutically acceptable enantiomer, pharmaceutically acceptable derivative, pharmaceutically acceptable polymorph or pharmaceutically acceptable prodrug thereof.

The method may be for making the formulation described in the first two aspects of the invention.

According to a fifth aspect of the present invention, there is provided a metered dose inhaler comprising a formulation according to any one of claims 1 to 18, and a canister coated with a polymer. The polymer may be selected from the group consisting of a fluorocarbon polymer, an epoxy copolymer, and an ethylene copolymer.

In an embodiment, the metered dose inhaler further comprises a sealing gasket. The sealing gasket may comprise a butyl elastomer.

According to a sixth aspect of the present invention, there is provided a method of treating bronchoconstriction, bronchospasm, asthma and related disorders comprising administering an effective amount of a formulation described above to patient in need thereof.

According to a seventh aspect of the present invention, there is provided the use of a formulation described above in medicine.

According to an eighth aspect of the present invention, there is provided the use of a formulation described above in the manufacture of a medicament for treating bronchoconstriction, bronchospasm, asthma and related disorders.

Thus, the present invention provides a stable metered dose inhaler (“MDI”) formulation comprising a pharmaceutically active agent with an HFA propellant along with suitable excipients. Surprisingly, it was found that a combination of particular active ingredients with particular excipients provides a stable MDI formulation. The active ingredients are an anticholinergic agent and a beta-agonist agent. The particular excipients are cosolvents other than alcohol, like polyethylene glycol (“PEG”), propylene glycol, isopropyl myristrate or glycerol, optionally in combination with a surfactant, and an HFA propellant and other suitable excipients. The formulation of the present invention does not form agglomerates. The present invention also provides a process for manufacture of a metered dose inhalation formulation. In an embodiment, the cosolvent is not an alcohol. In another embodiment, the cosolvent is not water.

It has also been surprisingly discovered that the use of polyoxyethylene sorbitan monolaurate and an HFA propellant and other suitable excipients, without an additional cosolvent, provides a stable formulation with the particular active ingredients.

The present invention also provides a method for the treatment of bronchoconstriction, bronchospasm, asthma and related disorders thereof, which method comprises administering to a patient in need thereof an effective amount of a metered dose inhalation formulation according to the present invention.

Other aspects of the invention will become apparent by consideration of the detailed description and examples.

### DETAILED DESCRIPTION

The present invention provides a stable aerosol pharmaceutical formulation. More specifically, the stable aerosol pharmaceutical formulation contains a pharmaceutically active agent in combination with a hydrofluoroalkane (“HFA”) propellant and other suitable excipients.

As discussed earlier, aerosol formulations traditionally contained CFC propellants. Due to environmental concerns, HFA propellants are now preferred over CFC propellants. As will be understood by those skilled in the art, suitable HFA propellants for use in the present invention include, but are not limited to, 1,1,1,2-tetrafluoroethane (HFA-134a) and 1,1,1,2,3,3,3-heptafluoropropane (HFA-227).

The formulations of the present invention are suitable for use in MDIs. MDIs are compact drug delivery systems that use a liquefied propellant to atomize a precisely metered volume of a pharmaceutical formulation into particles, which are small enough to penetrate deep into the patient's lungs. MDIs allow for targeted delivery of the drug to the desired site of the therapeutic effect—the lung.

The formulation of the present invention also includes a cosolvent, such as polyethylene glycol (“PEG”), propylene glycol, isopropyl myristrate or glycerol. Suitably, the cosolvent is PEG in liquid form, such as PEG 200 or PEG 400. The cosolvent can be present in a range of about 0.05% to about 15% by weight of the formulation. Suitably, the cosolvent is present in a range of about 0.05% to about 1% or about 0.05% to about 0.3% by weight of the formulation.

Further, it has been found that when an optional surfactant is used along with the cosolvent, the surfactant maintains the homogeneity of the suspension and also acts as a lubricant for the smooth functioning of the valve on the MDI. Suitable surfactants include, but are not limited to, polyvinylpyrrolidone (“PVP”), sorbitan trioleate, oleic acid, citric acid, and polyoxyethylene(4)lauryl ether (Brij 30®). Suitably the surfactant is PVP, such as PVP K25 or PVP K30 or PVP K17. The surfactant can be present in a range of about 0.00001% to about 10% by weight of the formulation. Suitably, the surfactant is present in a range of about 0.00001% to about 0.1% or about 0.0001% to about 0.001% by weight of the formulation.

Suitably, the formulations of the present invention are substantially free of water and alcohol. The term “substantially free of” means that the formulation contains less than about 5% water or alcohol by weight of the formulation. The formulations may contain less than about 3% water or alcohol. Preferably, the formulations contain less than about 1%, more preferably less than about 0.5%, still more preferably less than 0.1% or still more preferably less than about 0.05% water or alcohol. Most preferably, the formulations of the present invention contain no water or alcohol.

The present invention further provides a pharmaceutical formulation comprising an anticholinergic agent, a beta-agonist agent, polyoxyethylene sorbitan monolaurate, and a hydrofluoroalkane propellant. The polyoxyethylene sorbitan monolaurate can be present in a range of about 0.00001% to about 10% by weight of the formulation. Suitably, the polyoxyethylene sorbitan monolaurate is present in a range of about 0.00001% to about 0.1% or about 0.0001% to about 0.001% by weight of the formulation. More suitably, the polyoxyethylene sorbitan monolaurate is present in an amount of about 0.05% by weight of the formulation.

Pharmaceutically active agents useful in the formulations of the present invention include one or more of drugs selected from the class of beta-agonists agents and anticholinergic agents. The terms “beta-agonist agent” or “beta-agonist” or “anticholinergic agent” are used in a broad sense to include not only the beta-agonist or anticholinergic agent per se but also their pharmaceutically acceptable salts, pharmaceutically acceptable solvates and pharmaceutically acceptable hydrates. It will also be appreciated that the terms “beta-agonist agent” or “beta-agonist” or “anticholinergic agent” may also include pharmaceutically acceptable enantiomers, pharmaceutically acceptable derivatives, pharmaceutically acceptable enantiomers, pharmaceutically acceptable polymorphs, pharmaceutically acceptable prodrugs, etc.

Beta-agonist agents useful in the formulations of the present invention include, but are not limited to, albuterol, formoterol, levalbuterol, pirbuterol and salmeterol. The international name for albuterol is salbutamol. Suitable pharmaceutically acceptable salts of the beta-agonists include, but are not limited to the hydrochloride, sulfate, maleate, tartrate, and citrate salts. Suitably, the beta-agonist is albuterol or albuterol sulfate.

Anticholinergic agents useful in the formulations of the present invention include, but are not limited to, ipratropium and tiotropium. Suitable pharmaceutically acceptable salts of the anticholinergic agents include, but are not limited to, the halide salts such as bromide, chloride and iodide. Suitably, the anticholinergic agent is ipratropium or ipratropium bromide or ipratropium bromide monohydrate.

Suitably, the beta-agonist in the formulations is albuterol and the anticholinergic agent is ipratropium or ipratropium bromide or ipratropium bromide monohydrate. Alternatively, the beta-agonist in the formulations is albuterol sulfate and the anticholinergic agent is ipratropium or ipratropium bromide or ipratropium bromide monohydrate.

The present invention also provides a method of manufacturing a stable aerosol formulation according to the present invention. If used, the surfactant is dissolved in the cosolvent. The resulting solution is then mixed with an HFA propellant. If the surfactant is not used, the cosolvent is mixed with an HFA propellant. The pharmaceutically active agent is homogenized with additional HFA propellant to form a homogenized suspension. The homogenized suspension of active ingredients and solution of cosolvent and HFA propellant are mixed to form a second homogeneous suspension. The homogeneous second suspension is then placed in a precrimped canister or other container suitable for use as a metered dose inhaler.

For example, surfactant PVP K25 is dissolved in cosolvent PEG200 or PEG400 to make a clear solution. A quantity of HFA-227 propellant is added to the clear solution. A first homogenized suspension of ipratropium bromide and albuterol sulfate and additional HFA-227 propellant is prepared. The first homogenized suspension is added to the solution of PVP K25, PEG200 or PEG400 and HFA-227 to form a second homogeneous suspension. The resulting second suspension is then placed in a precrimped canister or other container suitable for use as a metered dose inhaler.

The present invention further provides a method for the treatment of bronchoconstriction, bronchospasm, asthma and related disorders thereof, comprising administering to a patient in need thereof a stable aerosol formulation according to the present invention. Related disorders include, but are not limited to, chronic obstructive pulmonary disease, chronic bronchitis and emphysema.

The formulation of the present invention may be administered one, two, three or four times per day with one or more activations, e.g. two, three or four activations, of the metering valve per administration to treat bronchoconstriction, asthma and related disorders thereof. Up to about twelve inhalations of the pharmaceutical formulation of the present invention may be administered per 24 hour period.

Suitably, each actuation of the metering valve delivers about 21 μg of an anticholinergic agent, such as ipratropium bromide monohydrate and about 120 μg of a beta-agonist agent, such as albuterol sulfate from the metered dose inhaler. Suitably, each container or metered dose inhaler canister contains about 200 inhalations. As one skilled in the art is aware, the dose may be adjusted depending on the therapeutic objective of the use of the active agents and the age and condition of the patient.

We observed that some aerosol drugs tend to adhere to the inner surfaces, i.e., walls of the cans and valves, of the MDI. This can lead to the patient getting significantly less than the prescribed amount of the active agent upon each activation of the MDI. Coating the inner surface of the container with a suitable polymer can reduce this adhesion problem. Suitable coatings include, but are not limited to, fluorocarbon copolymers such as FEP-PES (fluorinated ethylene propylene and polyethersulphone) and PFA-PES (perfluoroalkoxyalkane and polyethersulphone), epoxy and ethylene.

Also, during storage, moisture can enter the MDI mainly through the crimped area of the valve and through the stem by diffusion. To reduce the amount of moisture entering the MDI, the metering valve is suitably comprised of a butyl elastomer.

It will be readily apparent to one skilled in the art that varying substitutions and modifications may be made to the invention disclosed herein without departing from the spirit of the invention. Thus, it should be understood that although the present invention has been specifically disclosed by the preferred embodiments and optional features, modification and variation of the concepts herein disclosed may be resorted to by those skilled in the art, and such modifications and variations are considered to be falling within the scope of the invention.

The following examples are for the purpose of illustration of the invention only and are not intended in any way to limit the scope of the present invention.

### EXAMPLES

### Example 1

Formulations 1 to 18 listed below were prepared in the following manner.

The surfactant was dissolved in the cosolvent by sonication/homogenization for 10 minutes. This solution was mixed for 10-25 minutes in a mixing vessel with approximately 10%-15% of the total amount of propellant required for the batch. If the surfactant was not used, only the cosolvent was mixed with the propellant.

The active agents were homogenized in a separate homogenizer with approximately 5-15% of the total amount of propellant required for the batch. The homogenization speed and time ranged from 1000-1500 RPM for 15 to 30 minutes. The resulting homogenized suspension was transferred from the homogenizer to the mixing vessel through a double diaphragm pump. The remaining quantity of the propellant was then added to the mixing vessel.

The suspension was then mixed under constant stirring of 200-300 RPM for not less than 20 minutes and kept under recirculation through the double diaphragm pump.

Formulation 1

| | Qty/can | |
|---|---|---|
| Ipratropium Bromide monohydrate | 5.04 | mg |
| Albuterol Sulfate | 28.8 | mg |
| Sorbitan Trioleate 0.00002% | 0.00408 | mg |
| PEG 200 0.05% | 10.2 | mg |
| HFA-227 | 20.4 | gm |

Formulation 2

| | Qty/can | |
|---|---|---|
| Ipratropium Bromide monohydrate | 5.04 | mg |
| Albuterol Sulfate | 28.8 | mg |
| Sorbitan Trioleate 0.00004% | 0.00816 | mg |
| PEG 200 0.05% | 10.2 | mg |
| HFA-227 | 20.4 | gm |

Formulation 3

| | Qty/can | |
|---|---|---|
| Ipratropium Bromide monohydrate | 5.04 | mg |
| Albuterol Sulfate | 28.8 | mg |
| Sorbitan Trioleate 0.05% | 10.2 | mg |
| PEG 200 0.05% | 10.2 | mg |
| HFA-227 | 20.4 | gm |

Formulation 4

| | Qty/can | |
|---|---|---|
| Ipratropium Bromide monohydrate | 5.04 | mg |
| Albuterol Sulfate | 28.8 | mg |
| PVP K 25 0.00001% | 0.00204 | mg |
| PEG 200 0.1% | 20.4 | mg |
| HFA-227 | 20.4 | gm |

Formulation 5

| | Qty/can | |
|---|---|---|
| Ipratropium Bromide monohydrate | 5.04 | mg |
| Albuterol Sulfate | 28.8 | mg |
| PVP K 25 0.00001% | 0.00204 | mg |
| PEG 200 1% | 204 | mg |
| HFA-227 | 20.2 | gm |

Formulation 6

| | Qty/can | |
|---|---|---|
| Ipratropium Bromide monohydrate | 5.04 | mg |
| Albuterol Sulfate | 28.8 | mg |
| PVP K 25 0.001% | 0.204 | mg |
| PEG 200 0.3% | 61.2 | mg |
| HFA-227 | 20.2 | gm |

Formulation 7

| | Qty/can | |
|---|---|---|
| Ipratropium Bromide monohydrate | 5.04 | mg |
| Albuterol Sulfate | 28.8 | mg |
| PVP K 30 0.1% | 20.4 | mg |
| PEG 200 1% | 204 | mg |
| HFA-227 | 20.2 | gm |

Formulation 8

| | Qty/can | |
|---|---|---|
| Ipratropium Bromide monohydrate | 5.04 | mg |
| Albuterol Sulfate | 28.8 | mg |
| PVP K 30 0.001% | 0.204 | mg |
| PEG 200 0.3% | 61.2 | mg |
| HFA-227 | 20.3 | gm |

Formulation 9

| | Qty/can | |
|---|---|---|
| Ipratropium Bromide monohydrate | 5.04 | mg |
| Albuterol Sulfate | 28.8 | mg |
| Oleic acid 0.001% | 0.204 | mg |
| PEG200 1% | 204 | mg |
| HFA-227 | 20.2 | gm |

Formulation 10

| | Qty/can | |
|---|---|---|
| Ipratropium Bromide monohydrate | 5.04 | mg |
| Albuterol Sulfate | 28.8 | mg |
| Sorbitan Trioleate 0.00002% | 0.00408 | mg |
| PEG 400 0.05% | 10.2 | mg |
| HFA-227 | 20.4 | gm |

Formulation 11

| | Qty/can | |
|---|---|---|
| Ipratropium Bromide monohydrate | 5.04 | mg |
| Albuteol Sulfate | 28.8 | mg |
| Sorbitan Trioleate 0.00004% | 0.00816 | mg |
| PEG 400 0.05% | 10.2 | mg |
| HFA-227 | 20.4 | gm |

Formulation 12

| | Qty/can | |
|---|---|---|
| Ipratropium Bromide monohydrate | 5.04 | mg |
| Albuterol Sulfate | 28.8 | mg |
| Sorbitan Trioleate 0.05% | 10.2 | mg |
| PEG 400 0.05% | 10.2 | mg |
| HFA-227 | 20.4 | gm |

Formulation 13

| | Qty/can | |
|---|---|---|
| Ipratropium Bromide monohydrate | 5.04 | mg |
| Albuterol Sulfate | 28.8 | mg |
| PVP K 25 0.00001% | 0.00204 | mg |
| PEG 400 0.1% | 20.4 | mg |
| HFA-227 | 20.4 | gm |

Formulation 14

| | Qty/can | |
|---|---|---|
| Ipratropium Bromide monohydrate | 5.04 | mg |
| Albuterol Sulfate | 28.8 | mg |
| PVP K 25 0.00001% | 0.00204 | mg |
| PEG 400 1% | 204 | mg |
| HFA-227 | 20.2 | gm |

Formulation 15

| | Qty/can | |
|---|---|---|
| Ipratropium Bromide monohydrate | 5.04 | mg |
| Albuterol Sulfate | 28.8 | mg |
| PVP K 25 0.001% | 0.204 | mg |
| PEG 400 0.3% | 61.2 | mg |
| HFA-227 | 20.2 | gm |

Formulation 16

| | Qty/can | |
|---|---|---|
| Ipratropium Bromide monohydrate | 5.04 | mg |
| Albuterol Sulfate | 28.8 | mg |
| PVP K 30 0.1% | 20.4 | mg |
| PEG 400 1% | 204 | mg |
| HFA-227 | 20.2 | gm |

Formulation 17

| | Qty/can | |
|---|---|---|
| Ipratropium Bromide monohydrate | 5.04 | mg |
| Albuterol Sulfate | 28.8 | mg |
| PVP K 30 0.001% | 0.204 | mg |
| PEG 400 0.3% | 61.2 | mg |
| HFA-227 | 20.3 | gm |

Formulation 18

| | Qty/can | |
|---|---|---|
| Ipratropium Bromide monohydrate | 5.04 | mg |
| Albuterol Sulfate | 28.8 | mg |
| Oleic acid 0.001% | 0.204 | mg |
| PEG400 1% | 204 | mg |
| HFA-227 | 20.2 | gm |

### Example 2

Formulation 19 listed below was prepared in the following manner.

Polyoxyethylene sorbitan monolaurate (Tween 20) was dissolved in HFA propellant to form a solution. The active ingredients were homogenized with additional HFA propellant. The solution was then mixed with the homogenized suspension of active ingredients and HFA to form a second homogeneous suspension. The second suspension was then placed in a precrimped canister or other container suitable for use as an MDI. The formulation showed good stability.

Formulation 19

| | Qty/can | |
|---|---|---|
| Ipratropium Bromide monohydrate | 5.04 | mg |
| Albuterol Sulfate | 28.8 | mg |
| Tween 20 (0.05%) | 10.2 | mg |
| HFA-227 | 20.4 | gm |

### Example 3

Formulations made according to the process described above in Example 1 were tested for stability. Stability was determined by analyzing the particle size of the active ingredients using microscopy. The results are shown below in Table 1.

Examples 1 to 4 exemplify prior art formulations. Examples 5 to 7 exemplify the formulation of the present invention.

An increase in particle size in examples 1 to 4, indicated that the active ingredients were not as stable in the prior art formulations compared to the stability of the active ingredients in the formulations of the present invention.

TABLE 1
Effect of different combinations of cosolvent and surfactant on the suspension
characteristics and particle size of ipratropium bromide and albuterol sulfate.

| | Active | | | | Suspension | Particle size |
|---|---|---|---|---|---|---|
| | Ingredients | Cosolvent | Surfactant | Propellant | characteristics | observation |
| 1 | Ipratropium | — | — | HFA | Particles remain | Agglomeration |
| | (5.04 mg) | | | Propellant | in homogeneous | |
| | Albuterol | | | (P134a or | suspension for | |
| | sulfate | | | P227) | less than 5 | |
| | (28.8 mg) | | | | seconds. | |
| 2 | Ipratropium | Alcohol | — | HFA | Particles remain | 70% particles |
| | (5.04 mg) | (1-5%) | | Propellant | in homogeneous | between 10 to |
| | Albuterol | | | (P134a or | suspension for | 12.5 microns |
| | sulfate | | | P227) | about 10-15 | |
| | (28.8 mg) | | | | seconds. | |
| 3 | Ipratropium | Alcohol/ | — | HFA | Particles remain | 85% particles |
| | (5.04 mg) | Water | | Propellant | in homogeneous | between 10 to |
| | Albuterol | (1-5%) | | (P134a or | suspension for | 12.5 microns with |
| | sulfate | | | P227) | about 10-15 | agglomeration |
| | (28.8 mg) | | | | seconds. | |
| 4 | Ipratropium | Alcohol | Polyvinyl- | HFA | Particles remain | 70% particles |
| | (5.04 mg) | (1-5%) | pyrrolidone or | Propellant | in homogeneous | between 10 to |
| | Albuterol | | sorbitan | (P134a or | suspension for | 12.5 microns |
| | sulfate | | trioleate or | P227) | about 10-15 | |
| | (28.8 mg) | | oleic acid or | | seconds. | |
| | | | citric acid or | | | |
| | | | polyoxy- | | | |
| | | | ethylene(4) | | | |
| | | | lauryl ether | | | |
| | | | (0.02-10%) | | | |
| 5 | Ipratropium | PEG200/ | — | HFA | Particles remain | 90% particles |
| | (5.04 mg) | 400 | | Propellant | in homogeneous | below 2.5 microns |
| | Albuterol | (0.05-15%) | | (P134a or | suspension for | & 10% between |
| | sulfate | | | P227) | about 10-15 | 2.5 to 5 microns |
| | (28.8 mg) | | | | seconds. | |
| 6 | Ipratropium | PEG200/ | Polyvinyl- | HFA | Particles remain | 90% particles |
| | (5.04 mg) | 400 | pyrrolidoneor | Propellant | in homogeneous | below 2.5 microns |
| | Albuterol | (0.05-15%) | sorbitan | (P134a or | suspension for | & 10% between |
| | sulfate | | trioleate or | P227) | about 10-15 | 2.5 to 5 microns |
| | (28.8 mg) | | oleic acid or | | seconds. | |
| | | | citric acid or | | | |
| | | | polyoxy- | | | |
| | | | ethylene(4) | | | |
| | | | lauryl ether | | | |
| | | | (0.00001-10%) | | | |
| 7 | Ipratropium | — | Polysorbate 20 | HFA | Particles remain | 90% particles |
| | (5.04 mg) | | or Polysorbate | Propellant | in homogeneous | below 2.5 microns |
| | Albuterol | | 80 (0.01- | (P134a or | suspension for | & 10% between |
| | sulfate | | 0.05%) | P227) | about 10-15 | 2.5 to 5 microns |
| | (28.8 mg) | | | | seconds. | |

It is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

It must be noted that, as used in this specification and the appended claims, the singular forms “a,” “an” and “the” include plural references unless the context clearly dictates otherwise. Thus, for example, reference to “a propellant” includes a single propellant as well as two or more different propellants, reference to a “cosolvent” refers to a single cosolvent or to combinations of two or more cosolvents, and the like.

1. A pharmaceutical formulation comprising: an anticholinergic agent or a pharmaceutically acceptable salt, pharmaceutically acceptable solvate, pharmaceutically acceptable hydrate, pharmaceutically acceptable enantiomer, pharmaceutically acceptable derivative, pharmaceutically acceptable polymorph or pharmaceutically acceptable prodrug thereof; a beta-agonist agent or a pharmaceutically acceptable salt, pharmaceutically acceptable solvate, pharmaceutically acceptable hydrate, pharmaceutically acceptable enantiomer, pharmaceutically acceptable derivative, pharmaceutically acceptable polymorph or pharmaceutically acceptable prodrug thereof; a cosolvent selected from the group consisting of polyethylene glycol, propylene glycol, glycerol, and isopropyl myristrate; and a hydrofluoroalkane propellant.

2. The formulation according to claim 1, wherein the formulation further comprises a surfactant.

3. The formulation according to claim 2, wherein the surfactant is selected from the group consisting of polyvinylpyrrolidone, sorbitan trioleate, oleic acid, citric acid, and polyoxyethylene(4)lauryl ether.

4. The formulation according to claim 1, wherein the anticholinergic agent is selected from ipratropium or tiotropium or pharmaceutically acceptable salts, pharmaceutically acceptable solvates, pharmaceutically acceptable hydrates, pharmaceutically acceptable enantiomers, pharmaceutically acceptable derivatives, pharmaceutically acceptable polymorphs or pharmaceutically acceptable prodrugs thereof.

5. The formulation according to claim 4, wherein the anticholinergic agent is ipratropium.

6. The formulation according to claim 1, wherein the beta-agonist is selected from the group consisting of albuterol, formoterol, levalbuterol, pirbuterol and salmeterol, or pharmaceutically acceptable salts, pharmaceutically acceptable solvates, pharmaceutically acceptable hydrates, pharmaceutically acceptable enantiomers, pharmaceutically acceptable derivatives, pharmaceutically acceptable polymorphs or pharmaceutically acceptable prodrugs thereof.

7. The formulation according to claim 6, wherein the beta-agonist is albuterol.

8. The formulation according to claim 1, wherein the anticholinergic agent is ipratropium or a pharmaceutically acceptable salt, pharmaceutically acceptable solvate, pharmaceutically acceptable hydrate, pharmaceutically acceptable enantiomer, pharmaceutically acceptable derivative, pharmaceutically acceptable polymorph or pharmaceutically acceptable prodrug thereof and the beta-agonist is albuterol or a pharmaceutically acceptable salt, pharmaceutically acceptable solvate, pharmaceutically acceptable hydrate, pharmaceutically acceptable enantiomer, pharmaceutically acceptable derivative, pharmaceutically acceptable polymorph or pharmaceutically acceptable prodrug thereof.

9. The formulation according to claim 1, wherein the cosolvent is present in an amount of about 0.05% to about 15% of the total weight of the formulation.

10. The formulation according to claim 2, wherein the surfactant is present in an amount of about 0.00001% to about 10% of the total weight of the formulation.

11. The formulation according to claim 1, wherein the formulation is substantially free of alcohol.

12. The formulation according to claim 1, wherein the formulation is substantially free of water.

13. The formulation according to claim 1, wherein the formulation contains less than about 0.1% water by weight of the formulation.

14. The formulation according to claim 1, comprising ipratropium bromide or its monohydrate, albuterol sulfate, about 0.05% to about 1% polyethylene glycol, about 0.00001% to about 0.1% polyvinylpyrrolidone, and a hydrofluoroalkane propellant.

15. A pharmaceutical formulation comprising: an anticholinergic agent or a pharmaceutically acceptable salt, pharmaceutically acceptable solvate, pharmaceutically acceptable hydrate, pharmaceutically acceptable enantiomer, pharmaceutically acceptable derivative, pharmaceutically acceptable polymorph or pharmaceutically acceptable prodrug thereof; a beta-agonist agent or a pharmaceutically acceptable salt, pharmaceutically acceptable solvate, pharmaceutically acceptable hydrate, pharmaceutically acceptable enantiomer, pharmaceutically acceptable derivative, pharmaceutically acceptable polymorph or pharmaceutically acceptable prodrug thereof; a polysorbate; and a hydrofluoroalkane propellant, wherein there is no cosolvent present in the formulation.

16. The formulation according to claim 15, wherein the polysorbate is selected from polyoxyethylene (20) sorbitan monolaurate, polyoxyethylene (20) sorbitan monopalmitate, polyoxyethylene (20) sorbitan monostearate, polyoxyethylene (20) sorbitan monooleate and polyoxyethylene (20) sorbitan monoisostearate.

17. The formulation according to claim 15, wherein the polysorbate is polyoxyethylene (20) sorbitan monolaurate.

18. The formulation according to claim 15, wherein the polysorbate is present in an amount of about 0.05% by weight of the formulation.

19. A method of making a pharmaceutical formulation comprising:
(a) mixing a hydrofluoroalkane propellant with a cosolvent to form a solution;
(b) forming a first homogenized suspension of an anticholinergic agent or a pharmaceutically acceptable salt, pharmaceutically acceptable solvate, pharmaceutically acceptable hydrate, pharmaceutically acceptable enantiomer, pharmaceutically acceptable derivative, pharmaceutically acceptable polymorph or pharmaceutically acceptable prodrug thereof; a beta-agonist agent or a pharmaceutically acceptable salt, pharmaceutically acceptable solvate, pharmaceutically acceptable hydrate, pharmaceutically acceptable enantiomer, pharmaceutically acceptable derivative, pharmaceutically acceptable polymorph or pharmaceutically acceptable prodrug thereof; and a hydrofluoroalkane propellant; and
(c) adding the first homogenized suspension to the solution to form a second homogeneous suspension.

20. The method according to claim 19, wherein the cosolvent is selected from the group consisting of polyethylene glycol, propylene glycol, glycerol, and isopropyl myristrate.

21. The method according to claim 19, further comprising dissolving a surfactant in the cosolvent.

22. The method according to claim 21, wherein the surfactant is selected from the group consisting of polyvinylpyrrolidone, sorbitan trioleate, oleic acid, citric acid, and polyoxyethylene(4)lauryl ether.

23. A method of making a pharmaceutical formulation comprising:
(a) dissolving a polysorbate in a hydrofluoroalkane propellant to form a solution;
(b) forming a first homogenized suspension of an anticholinergic agent or a pharmaceutically acceptable salt, pharmaceutically acceptable solvate, pharmaceutically acceptable hydrate, pharmaceutically acceptable enantiomer, pharmaceutically acceptable derivative, pharmaceutically acceptable polymorph or pharmaceutically acceptable prodrug thereof; a beta-agonist agent or a pharmaceutically acceptable salt, pharmaceutically acceptable solvate, pharmaceutically acceptable hydrate, pharmaceutically acceptable enantiomer, pharmaceutically acceptable derivative, pharmaceutically acceptable polymorph or pharmaceutically acceptable prodrug thereof; and a hydrofluoroalkane propellant; and
(c) adding the first homogenized suspension to the solution to form a second suspension.

24. The method according to claim 23, wherein the polysorbate is selected from polyoxyethylene (20) sorbitan monolaurate, polyoxyethylene (20) sorbitan monopalmitate, polyoxyethylene (20) sorbitan monostearate, polyoxyethylene (20) sorbitan monooleate and polyoxyethylene (20) sorbitan monoisostearate.

25. The method according to claim 23, wherein the polysorbate is polyoxyethylene (20) sorbitan monolaurate

26. A metered dose inhaler comprising a formulation according to claim 1 and a canister coated with a polymer.

27. The metered dose inhaler according to claim 26, wherein the polymer is selected from the group consisting of a fluorocarbon polymer, an epoxy copolymer, and an ethylene copolymer.

28. The metered dose inhaler according to claim 26, further comprising a sealing gasket.

29. The metered dose inhaler according to claim 28, wherein the sealing gasket comprises a butyl elastomer.

30. A method of treating bronchoconstriction, bronchospasm, asthma and related disorders comprising administering an effective amount of a formulation according to claim 1 to patient in need thereof.

31. A method comprising using a formulation according to claim 1 in medicine.

32. A method comprising using a formulation according to claim 1 in the manufacture of a medicament for treating bronchoconstriction, bronchospasm, asthma and related disorders.
